(19) United States
(12) Patent Application Publication
(10) Pub. No.: US 2026/0321403 A1
(43) Pub. Date: Oct. 15, 2026

(54) RARE DISEASE DIAGNOSIS AND ACCESS INTEGRATED PLATFORM

(71) Applicant: Chandrashekhar Sattiraju, Westwood, MA (US)

(72) Inventor: Chandrashekhar Sattiraju, Westwood, MA (US)

(21) Appl. No.: 19/197,970

(22) Filed: May 3, 2025

Related U.S. Application Data
(60) Provisional application No. 63/786,308, filed on Apr. 10, 2025.
(60) Provisional application No. 63/791,504, filed on Apr. 19, 2025.
(60) Provisional application No. 63/791,511, filed on Apr. 19, 2025.
(60) Provisional application No. 63/791,514, filed on Apr. 19, 2025.
(60) Provisional application No. 63/791,489, filed on Apr. 19, 2025.
(60) Provisional application No. 63/791,517, filed on Apr. 20, 2025.
(60) Provisional application No. 63/791,520, filed on Apr. 20, 2025.
(60) Provisional application No. 63/795,358, filed on Apr. 26, 2025.
(60) Provisional application No. 63/795,360, filed on Apr. 26, 2025.
(60) Provisional application No. 63/795,362, filed on Apr. 26, 2025.

(51) Int. Cl. G16H 40/20; G06F 21/12; G16H 10/60; G16H 20/10; G16H 50/30

(52) U.S. Cl. CPC G16H 40/20; G06F 21/123; G16H 10/60; G16H 20/10; G16H 50/30

(57) ABSTRACT

The disclosed invention relates to a modular, AI-enhanced computerized platform designed to improve rare disease diagnosis, clinical trial matching, financial aid discovery, and continuous access navigation. The system is configured to calculate triage risk scores, map eligibility pathways in real time, validate eligibility updates through hash-chained commit-lock auditing stored on a permissioned blockchain, and maintain uninterrupted patient guidance via latency-triggered fallback logic that reroutes navigation upon API latency events. By integrating fallback-logic pathways, edge-node secondary caching, and commit-lock blockchain auditing, the platform is architected to enhance audit integrity, reduce session interruptions, and based on theoretical modeling is expected to improve overall system responsiveness relative to conventional static or single-path access systems.

### CROSS REFERENCE TO RELATED APPLICATIONS

This application claims the benefit under 35 U.S.C. § 119(e) of the following U.S. Provisional Applications:
63/786,308, filed Apr. 10, 2025
63/791,504, filed Apr. 19, 2025
63/791,511, filed Apr. 19, 2025
63/791,514, filed Apr. 19, 2025
63/791,517, filed Apr. 20, 2025
63/791,520, filed Apr. 20, 2025
63/791,489, filed Apr. 19, 2025
63/795,358, filed Apr. 26, 2025
63/795,360, filed Apr. 26, 2025
63/795,362, filed Apr. 26, 2025

The disclosures of the aforementioned provisional applications are incorporated herein by reference in their entirety.

### BACKGROUND

Rare disease patients face significant challenges, including prolonged diagnostic delays, fragmented access to approved treatments, limited discovery of available financial aid programs, and underrepresentation in clinical research studies. Existing digital health tools are frequently static, lack adaptability to real-time eligibility changes, and are siloed across disconnected service providers—leading to inefficiencies and missed opportunities for coordinated patient support.

As a result, there remains a persistent need for a dynamic, interoperable digital health infrastructure capable of enabling resilient, real-time navigation across diagnostic, therapeutic, and research-related pathways. A system that can address these limitations by supporting live eligibility integration and cross-domain resource coordination would offer the potential to accelerate diagnosis, improve equitable access to services, and enhance research inclusion for rare disease populations.

### SUMMARY OF THE INVENTION

Unlike prior art systems that address rare disease diagnosis, patient navigation, or audit logging in isolation, the disclosed invention uniquely integrates (i) AI-based symptom triage, (ii) commit-lock blockchain auditing, and (iii) latency-triggered fallback logic into a cohesive, real-time eligibility navigation system. This integration addresses a long-standing unmet need for uninterrupted, tamper-evident access coordination in rare disease care, particularly under unstable network conditions and intermittent API failure.

The combination of these distinct technologies, each traditionally applied in separate domains results in a synergistic architecture that materially improves system resilience, audit verifiability, and real-time responsiveness. These improvements are not achievable by merely aggregating known tools or techniques, but arise from a deliberately engineered interaction between AI-driven triage logic, cryptographically verifiable eligibility state transitions, and autonomous fallback pathways that activate during latency spikes or query failures.

As such, the claimed invention provides a non-obvious technical improvement to computer functionality in the field of distributed medical informatics. It replaces static, single-path eligibility systems with a fault-tolerant, self-recovering platform capable of maintaining uninterrupted access navigation even during infrastructure disruptions solving technical challenges that remain unresolved in the current art.

The disclosed invention provides a modular, AI-enhanced computerized platform designed to improve the process of patient triage, clinical trial matching, and treatment access especially for patients with rare diseases. The system is composed of:
1. A Real-Time Crosslinked Navigation Engine;
2. A Community-Triggered Trial Recruitment Engine;
3. An Autonomous Data Enrichment and Commit-Lock Auditing Module; and
4. A Fallback Logic Framework ensuring continuous system operation even when eligibility matches are unavailable.

The platform is structured to address key technical challenges in medical informatics by improving real-time responsiveness, ensuring continuity of navigation under latency stress, and securing the auditability of eligibility decisions through cryptographic validation. The fallback subsystem is configured to automatically reroute navigation requests when latency thresholds are exceeded, while the commit-lock auditing module generates tamper-evident records of all eligibility updates, thereby reducing rollback vulnerabilities inherent in conventional database systems.

In particular, the system architecture combines edge-node secondary caching with a permissioned blockchain ledger that is validated by distributed validator nodes using a Practical Byzantine Fault Tolerance (PBFT) protocol or an equivalent consensus mechanism appropriate for permissioned blockchain environments (e.g., Raft, Paxos, or other fault-tolerant protocols). This dual-pathway configuration enhances network resilience and audit integrity.

When latency exceeds a configurable threshold, the platform is designed to retrieve pre-stored eligibility-vector snapshots from edge nodes, preserving the continuity of user navigation. In one embodiment, the system is configured to cryptographically hash all eligibility vector updates and append them to the blockchain ledger only after validator approval.

The system is configured to achieve high service availability. Based on theoretical modeling and system design assumptions, architecture is expected to reduce the risk of data-integrity compromise by approximately tenfold (10×) and to improve session continuity by up to ninety-five percent (95%) relative to conventional platforms that rely solely on centralized SQL audit tables and single-path API calls. These anticipated improvements are based on system-level architectural projections and have not yet been validated through live deployment.

In its intended configuration, the system operates autonomously, using AI-based data enrichment agents to update trial criteria, financial support data, and treatment pathways in the background. All eligibility matches and updates are routed through both fallback detection and commit-lock validation modules to ensure uninterrupted, high-integrity eligibility navigation.

This platform materially enhances the fault tolerance, audit reliability, and real-time performance of rare disease access systems. As outlined in MPEP § 2106.04, the disclosed system represents a concrete improvement to computer functionality, particularly in reducing eligibility processing latency and increasing verifiability of navigation outputs across distributed healthcare networks.

### BRIEF DESCRIPTION OF THE DRAWINGS

The figures are schematic and simplified for explanatory clarity; not all sub-components are illustrated, and the relative shapes and positions of modules are not drawn to scale.

FIG. 1: System-flow diagram showing data ingestion, triage scoring, navigation routing, trial matching, commit-lock auditing, fallback outputs.

FIG. 2: Community-monitoring loop detailing discussion ingestion, HPO tagging, eligibility scoring, and trial recommendation.

FIG. 3: 24-hour rolling window refresh of eligibility vector and commit-lock blockchain write.

FIG. 4: Fallback-trigger logic rerouting navigation after eligibility failures or latency timeouts.

### Definitions. As used herein:

As used herein:
1. [0014] Eligibility Vector: A structured data object-such as a JSON document or equivalent format-keyed by program identifiers, each associated with a Boolean eligibility outcome. The eligibility vector may be refreshed on a scheduled or event-driven basis (e.g., once every 24-hour period) and may be used to determine active pathways available to a patient.
2. [0015] Fallback Trigger: A system-defined event that initiates fallback rerouting in response to: (i) a condition where no affirmative eligibility matches are found within the eligibility vector; or (ii) a detected round-trip API latency that exceeds a configurable threshold (e.g., two seconds). Fallback rerouting may retrieve pre-stored eligibility-vector snapshots from edge-node secondary caches to maintain uninterrupted patient guidance
3. [0016] Access-Risk Score: A system-generated metric representing the likelihood that a patient may experience difficulty accessing treatments, based on a combination of urgency level, financial indicators, and eligibility outcomes across available programs.
4. [0017] Eligibility Query: A dynamic programmatic request, issued by the platform, to evaluate a patient's mapped phenotype codes, risk scores, or input data against live eligibility rules from treatment, financial, or research support programs.
5. [0018] Edge-Node Secondary Cache: A geographically distributed cache located at content-delivery edge nodes, configured to store anonymized eligibility-vector snapshots for low-latency re-query operations during times when the primary database is unavailable or experiences degraded performance.
6. [0019] Distributed Consensus Protocol: A cryptographic validation process through which eligibility vector updates are reviewed by multiple independent validator nodes (e.g., using Practical Byzantine Fault Tolerance), requiring a threshold consensus (e.g., two-thirds majority) before such updates are committed to a permissioned blockchain ledger.

### DETAILED DESCRIPTION

### Detailed Description of the Drawings

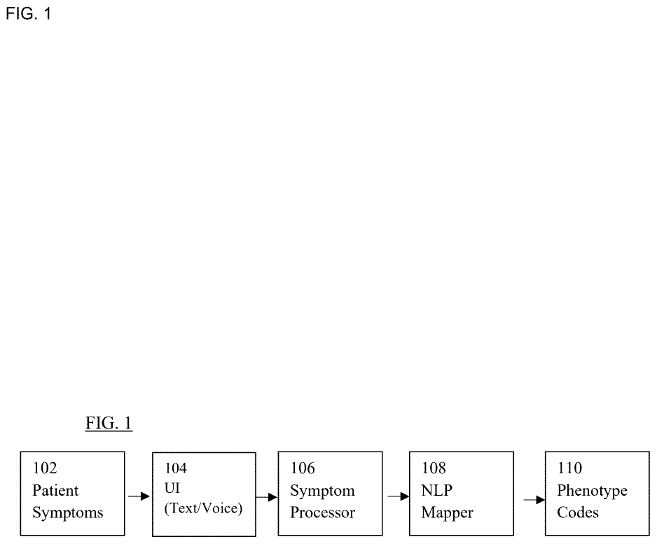

### FIG. 1

Referring to FIG. 1, patient symptoms 102 are entered via a user interface (UI) 104 supporting text or voice input. A symptom-processing module 106 then invokes an NLP mapper 108, which outputs standardized phenotype codes 110 (e.g., Human Phenotype Ontology (HPO) terms). These standardized codes are referred to herein as mapped phenotype codes. The mapped phenotype codes feed into a triage engine 112, which generates urgency scores and access-risk scores 114 based on the patient's reported symptoms. A navigation engine 116 receives the scores and phenotype codes, consults live eligibility vectors 118, and dynamically produces personalized pathways 120 that may include treatment access options 122, financial aid programs 124, and clinical-trial matches 126. All selected pathways are initially written to a staging log 128. Before finalization, each pathway selection is subjected to a commit-lock auditing process 130, which generates a cryptographic timestamp 132 to ensure tamper-evident eligibility validation. Eligibility updates are only committed after validation by the distributed commit-lock system. If an eligibility vector query returns null results, or if the round-trip latency for a query exceeds a configured threshold (e.g., two seconds), a fallback logic module 134 is triggered. This module surfaces secondary resources 136, such as specialist directories or educational guidance, to maintain uninterrupted patient support. The entire architecture operates on distributed processors 140 connected via a secure network 142. In some embodiments, the architecture further includes an edge-node secondary cache deployed at content-delivery edge servers to store anonymized eligibility-vector snapshots. These snapshots enable low-latency re-query during periods when the primary program API exhibits elevated latency, ensuring high system availability and resilience. The modular design allows each engine and module to operate independently while maintaining cohesive, real-time patient guidance across the system. This schematic illustrates one possible system architecture. Other deployment topologies may be used.

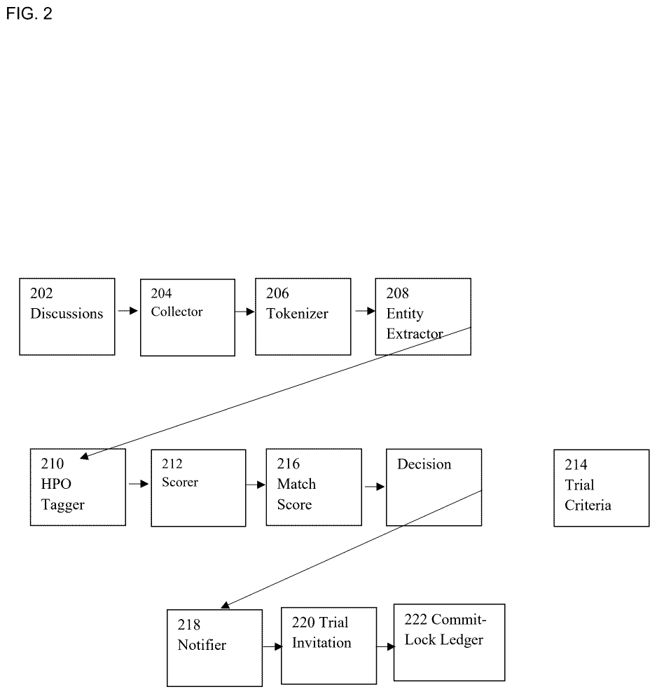

### FIG. 2

Referring to FIG. 2, a community-monitoring loop 200 is illustrated. Patient-generated discussions 202 from forums and social feeds are ingested by a streaming collector 204. The discussions are processed by a tokenizer 206 and an entity extractor 208 to identify candidate medical entities. These entities are mapped by an HPO tagger 210 to standardized phenotype codes. The mapped codes are evaluated by a community-eligibility scorer 212, which generates a match score 216 by comparing the extracted entities against live clinical trial criteria 214. When a match score exceeds a configured threshold, a recruitment notifier 218 dispatches a trial invitation 220 to the originating user profile. Each notification event is permanently logged in a commit-lock ledger 222 to ensure tamper-evident auditability.

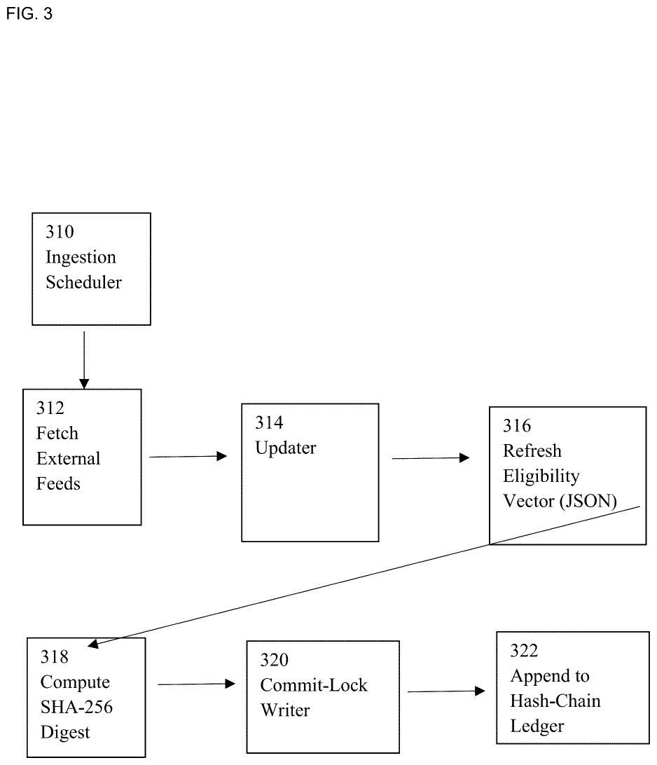

### FIG. 3

Referring to FIG. 3, an ingestion scheduler 310 fetches external program and trial feeds 312 once during each 24-hour rolling window. An updater 314 merges the results into a JSON eligibility-vector store 316, replacing stale keys and stamping each element with an RFC-3339 timestamp. A digest generator 318 computes a SHA-256 hash of the refreshed vector, and a commit-lock writer 320 appends the hash to a permissioned blockchain ledger 322, thereby creating an immutable, tamper-evident audit entry.

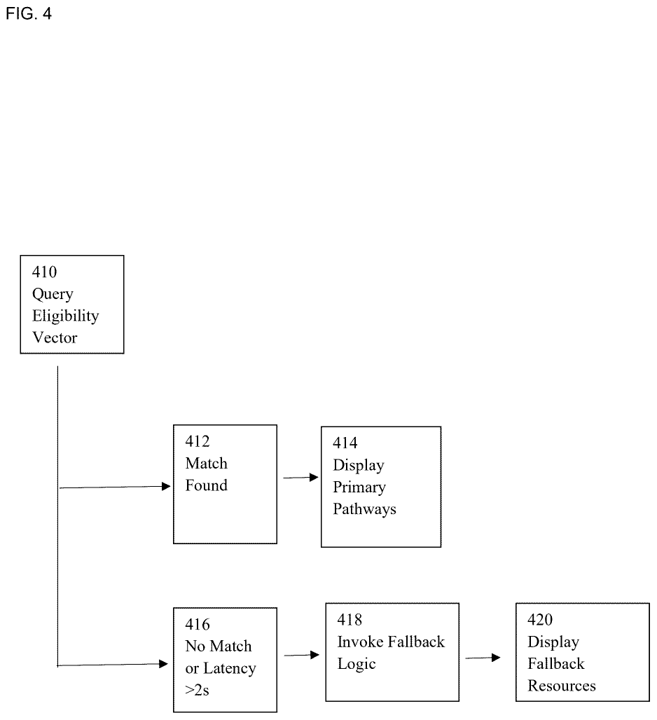

### FIG. 4

Referring to FIG. 4, the navigation engine submits a primary eligibility query 410. If the query returns one or more affirmative programs (branch 412), the system displays primary pathways 414. Otherwise—or if the round-trip API-request latency exceeds two seconds (decision 416)—a fallback logic module 418 automatically routes the user to secondary resources 420 such as specialist directories, educational materials, or generalized financial-aid programs, thus maintaining uninterrupted guidance even under upstream failure conditions. Fallback logic is triggered upon either (i) zero affirmative eligibility matches or (ii) exceeding API response latency thresholds.

### System Overview:

The disclosed platform leverages a modular, AI-enhanced computerized architecture that is designed to improve system resilience, ensure tamper-evident recordkeeping, and minimize patient navigation disruptions caused by network latency or eligibility lookup failures

The following subsections describe the core functional modules and their interplay in enabling a resilient, high-availability eligibility navigation system. In a preferred embodiment, these modules may be implemented using existing cloud infrastructure platforms such as AWS Lambda or Supabase Edge Functions. However, the invention is not limited to any specific provider or environment, and may be realized using any serverless or distributed computing architecture capable of supporting the described functionality.

### Commit-lock Auditing:

A commit-lock auditing process ensures that all eligibility matches or updates are recorded in a reversible validation layer with cryptographic timestamps before final commitment to the platform database. In one embodiment, the commit-lock auditing module operates as follows: (i) retrieve the updated eligibility-vector data structure (e.g., JSON object or equivalent format); (ii) compute a cryptographic hash digest of the content using a secure algorithm such as SHA-256, SHA-3, or an equivalent method providing collision resistance and tamper detection; (iii) transmit the digest to at least three validator nodes operating a distributed consensus protocol, such as a Practical Byzantine Fault Tolerance (PBFT) algorithm or an equivalent mechanism suitable for permissioned blockchain environments; (iv) await a minimum two-thirds majority validation; and (v) append the validated digest to a permissioned blockchain ledger. Raw patient data remain off-chain; only digests are recorded. This ensures tamper-evident auditability, prevents unauthorized rollback, and preserves the historical integrity of patient eligibility records. This commit-lock framework is intended to materially improve audit integrity in medical informatics systems by replacing conventional mutable logs with cryptographically verifiable, tamper-evident records.

### Placeholder Blocking:

Placeholder blocking mechanisms use data-type validators and completeness checks to prevent incomplete, ambiguous, or unverifiable entries from influencing eligibility workflows. In one embodiment, only records satisfying all required schema fields and matching at least one external eligibility rule are passed to the triage engine.

### Dynamic Crosslinking:

The Real-Time Crosslinked Navigation Engine continuously updates access pathways in response to changing patient inputs (e.g., updated symptoms or diagnoses) and live program eligibility feeds. By recalculating triage vectors and eligibility snapshots in near real-time, the system prevents outdated or invalid pathway recommendations and ensures up-to-date guidance.

### Human Phenotype Ontology (HPO) Mapping:

Patient symptoms are mapped using Natural Language Processing (NLP) techniques to structured phenotype codes such as Human Phenotype Ontology (HPO) entries. This transformation enables machine-readable indexing and downstream eligibility scoring not possible with unstructured narrative inputs.

### Daily Eligibility Vector Updates:

Eligibility vectors are refreshed once per 24-hour rolling window using structured API or RSS-based feeds from clinical trial registries, treatment authorizations, and nonprofit financial support databases. Each refresh operation appends a cryptographic timestamp and replaces stale entries to ensure current, verifiable eligibility matching.

### Consent Management System:

The system includes a consent management module that electronically captures, timestamps, and stores patient consent before initiating any clinical trial pre-screening communication. Consent is recorded in compliance with HIPAA and GDPR standards, and optionally linked to patient-reported symptoms or triage scores for downstream eligibility matching.

### Autonomous Data Enrichment:

Autonomous data enrichment refers to continuous background processing by AI agents that retrieve, parse, and structure external eligibility data (e.g., trial protocols, copay changes, grant availability). These enrichment processes use pattern matching, metadata tagging, and vector normalization to ensure accurate, machine-readable integration into eligibility vectors without interrupting live patient sessions.

### Fallback Logic:

Fallback logic is dynamically triggered upon either zero affirmative matches or a measured round-trip API latency exceeding a configurable threshold (e.g., two seconds). This adaptive rerouting prevents session timeouts and is designed to support high system availability (e.g., 99.999%) by rerouting user sessions. Fallback resources may include pre-fetched pathway snapshots, static financial directories, or educational material caches.

### Working Example 1:

The following examples illustrate intended system behavior and are presented for explanatory purposes only. The invention is not limited to these specific workflows
Jane Doe inputs symptoms via mobile application.
NLP engine maps symptoms to standardized HPO terms.
Triage engine generates urgency and access-risk scores.
Navigation engine dynamically matches mapped phenotype codes and urgency scores to trial and treatment eligibility pathways.
Eligibility matches are verified and committed via hash-chained commit-lock auditing to a permissioned blockchain ledger.
If no matches are found or latency exceeds two seconds, fallback logic reroutes Jane to specialist directories and financial aid resources without session timeout.
This example demonstrates improved system uptime, tamper-evident eligibility recordation, and continuous patient navigation compared to conventional static systems.

### Working Example 2-Trial Scoring and Blockchain Audit:

For each clinical trial:
Initialize score=0;
Increment score for each satisfied inclusion criterion;
Decrement score for each exclusion conflict;
Rank and recommend trials based on scores.
   if latency>2 seconds or no match found:
   trigger fallback routing
   else:
   commit eligibility match to blockchain ledger

The distributed consensus used to finalize each commit-lock entry may employ a Practical Byzantine Fault Tolerance (PBFT) protocol or any equivalent consensus algorithm suitable for permissioned blockchain environments (e.g., Raft, Paxos, or other fault-tolerant consensus protocols), executed across at least three validator nodes. This ensures cryptographic validation and tamper-evident auditability of eligibility transactions.

Collectively, the described modules and workflows reflect an architectural design intended to improve system availability, audit integrity, and eligibility-matching precision relative to conventional static or sequential systems. By integrating latency-based decision logic, cryptographic commit validation, and NLP-driven phenotype mapping, the system is structured to enable more responsive and verifiable patient navigation capabilities.

1. INDEPENDENT claim 1 (System Claim)
A system comprising:
a) distributed processors configured with:
an edge-node secondary cache; and
a permissioned blockchain commit-lock auditor validated by at least three validator nodes operating a-di-a distributed consensus protocol configured for fault-tolerant validation, including but not limited to Practical Byzantine Fault Tolerance (PBFT), Raft, Paxos, or any equivalent consensus mechanism capable of achieving quorum-based validation in permissioned blockchain environments;
b) a non-transitory memory storing instructions that, when executed by the processors, cause the system to:
receive patient symptom inputs;
map the patient symptom inputs to mapped phenotype codes;
generate urgency and access-risk scores based on mapped phenotype codes;
map the urgency scores and mapped phenotype codes to multiple pathways, including treatment access, clinical-trial enrollment, and financial support, by re-issuing an eligibility query to the edge-node secondary cache configured to store anonymized eligibility-vector snapshots for rapid re-query when a primary eligibility-vector request latency exceeds a threshold of two seconds;
match patient discussions to live clinical-trial eligibility criteria;
verify all eligibility vector updates using a hash-chained commit-lock auditor that:
(i) computes a cryptographic hash digest using a secure algorithm such as SHA-256, SHA-3, or an equivalent hash function capable of providing collision resistance, forward secrecy, and tamper-evident integrity as required for audit validation; and
(ii) appends the digest to a permissioned blockchain ledger replicated across at least three validator nodes; and
maintain continuous operation by triggering fallback-logic pathways when:
(i) no affirmative eligibility matches exist in the eligibility vector; or
(ii) a round-trip latency for an eligibility query exceeds two seconds,
wherein fallback rerouting retrieves eligibility-vector snapshots from the edge-node secondary cache.
Dependent Claims to claim 1:

2. The system of claim 1, wherein the eligibility vectors are updated at least daily based on external clinical-trial and treatment-authorization data feeds.

3. The system of claim 1, wherein:
an eligibility vector structured in a machine-readable serialization format—such as JSON, YAML, Protocol Buffers, FHIR, or any equivalent format—used to represent structured eligibility records, is refreshed at least every twenty-four hours or in response to external program changes;
the updates are validated by at least three validator nodes operating a distributed fault-tolerant consensus protocol—such as Practical Byzantine Fault Tolerance (PBFT), Raft, Paxos, or an equivalent mechanism—capable of achieving quorum-based validation in a permissioned blockchain environment before being appended to a permissioned blockchain ledger; and
fallback is triggered when (i) no affirmative matches exist in the refreshed eligibility vector or (ii) API latency exceeds a configurable threshold.

4. The system of claim 1, wherein placeholder data is blocked from eligibility-evaluation workflows until verification is complete.

5. The system of claim 1, wherein the commit-lock auditing mechanism records each data update in a cryptographic hash chain stored on a distributed ledger.

6. The system of claim 1, further comprising:
an eligibility-vector store containing entries structured in a machine-readable serialization format such as JSON, YAML, Protocol Buffers, FHIR, or any equivalent format used to represent structured eligibility records;
a hash-chained commit-lock auditor configured to generate a cryptographic hash digest using a secure algorithm-such as SHA-256, SHA-3, or an equivalent hash function-capable of providing collision resistance, forward secrecy, and tamper-evident integrity, and to record the digest on a permissioned blockchain ledger; and
a fallback logic module configured to reroute a patient request to secondary resources when (i) the eligibility-vector store contains no affirmative entries or (ii) a program-API request latency exceeds two seconds.

7. The system of claim 1, further comprising a means for rerouting patient navigation requests when (i) no affirmative eligibility match exists in the eligibility vector or (ii) the round-trip latency for a primary eligibility query exceeds a threshold,
wherein said means is configured to retrieve eligibility-vector snapshots from an edge-node secondary cache, and wherein the corresponding structure for performing the rerouting function includes latency detection modules, fallback pathway logic, and edge-cache re-query mechanisms as described in the specification.

8. The system of claim 1, further comprising a means for validating and committing eligibility-vector updates to a tamper-evident ledger, wherein said means comprises:
(i) a cryptographic hashing component configured to compute a digest of the eligibility vector using a secure algorithm such as SHA-256, SHA-3, or an equivalent;
(ii) a distributed consensus validator mechanism comprising at least three validator nodes operating a quorum-based protocol such as PBFT, Raft, Paxos, or equivalent; and
(iii) a permissioned blockchain ledger configured to receive and store the validated digest, as described in the specification.

9. INDEPENDENT claim 2 (Triage+Navigation Subsystem)
A system comprising a non-transitory memory and one or more processors configured to:
a) receive patient symptom inputs;
b) map the symptom inputs to standardized phenotype codes;
c) generate urgency scores based on mapped phenotype codes;
d) dynamically identify and update eligibility pathways based on the urgency scores and phenotype codes,
wherein the eligibility pathway mappings are refreshed in real time using updated eligibility vectors that are validated by a commit-lock auditing mechanism employing a permissioned blockchain ledger.
Dependent Claim to claim 2:

10. The system of claim 2, wherein the eligibility pathway mapping is dynamically prioritized based on urgency scores generated by the triage-risk calculation engine.

11. INDEPENDENT claim 3 (Community-Triggered Trial Matching Subsystem)
A system comprising a non-transitory memory and one or more processors configured to:
a) ingest unstructured patient-generated discussions from forums or social media feeds;
b) tokenize and extract medical entities from the ingested text;
c) map the extracted entities to standardized phenotype ontology codes;
d) compare the mapped codes against stored clinical trial eligibility criteria to identify candidate matches; and
e) generate and dispatch trial participation recommendations to associated user profiles based on the identified matches.
Dependent Claim to claim 3:

12. The system of claim 3, wherein the mapping step is performed by a tagging engine that uses natural-language processing to match layperson symptom phrases to standardized phenotype ontology codes.

13. INDEPENDENT claim 4 (Method Claim)
A computer-implemented method comprising:
a) receiving patient symptom inputs via a user interface;
b) mapping the symptom inputs to standardized phenotype codes;
c) generating triage urgency scores based on the mapped phenotype codes;
d) matching the urgency scores and phenotype codes to one or more actionable eligibility pathways;
e) validating the eligibility vector using a commit-lock auditing mechanism configured to compute a cryptographic hash digest using a secure algorithm—such as SHA-256, SHA-3, or an equivalent hash function-capable of providing collision resistance, forward secrecy, and tamper-evident integrity, and to append the digest to a permissioned blockchain ledger following validation by a distributed consensus protocol; and
f) rerouting the user to fallback resources when either (i) no affirmative eligibility matches are found, or (ii) a round-trip latency threshold for the eligibility query is exceeded.
Dependent Claims to claim 4:

14. The method of claim 4, wherein the fallback resources comprise at least one of a specialist referral directory or a set of educational guidance materials.

15. The method of claim 4, further comprising electronically capturing patient consent prior to initiating any clinical trial pre-screening invitation.

16. INDEPENDENT claim 5 (Computer-Readable Medium Claim)
A non-transitory computer-readable medium storing instructions that, when executed by one or more processors, cause the system to:
a) receive patient symptom inputs;
b) map the received inputs to standardized phenotype ontology codes;
c) dynamically match the mapped codes to one or more actionable eligibility pathways;
d) validate eligibility vector integrity using a hash-chained commit-lock auditing mechanism that computes a cryptographic hash digest using a secure algorithm such as SHA-256, SHA-3, or an equivalent hash function capable of providing collision resistance, forward secrecy, and tamper-evident integrity, and appends the digest to a permissioned blockchain ledger; and
e) trigger fallback resource rerouting when (i) no affirmative eligibility matches are detected in the eligibility vector, or (ii) a round-trip latency threshold is exceeded during the eligibility query process.
